(19) United States
(12) Patent Application Publication
(10) Pub. No.: US 2022/0315550 A1
(43) Pub. Date: Oct. 6, 2022

(54) SIMULTANEOUS DEHYDRATION OF GLUCOSE AND XYLOSE TO FURFURALS USING HETEROGENOUS SOLID ACID CATALYSTS

(71) Applicant: Alliance for Sustainable Energy, LLC, Golden, CO (US)

(72) Inventors: Ashutosh MITTAL, Lakewood, CO (US); David Kenneth JOHNSON, Denver, CO (US)

(21) Appl. No.: 17/666,493

(22) Filed: Feb. 7, 2022

Related U.S. Application Data
(60) Provisional application No. 63/146,520, filed on Feb. 5, 2021.

(51) Int. Cl. C07D 307/50; B01J 21/12; B01J 39/05

(52) U.S. Cl. CPC C07D 307/50; B01J 21/12; B01J 39/05

(57) ABSTRACT

Disclosed herein are the use of methods and compositions for the simultaneous dehydration of glucose and xylose present in a process relevant biorefinery hydrolysate to furfurals using heterogenous solid acid catalysts.

### CROSS-REFERENCE TO RELATED APPLICATIONS

This application claims priority under 35 U.S.C. § 119 to U.S. provisional patent application No. 63/146,520 filed on 05Feb.2021, the contents of which are hereby incorporated in their entirety.

### CONTRACTUAL ORIGIN

The United States Government has rights in this invention under Contract No. DE-AC36-08G028308 between the United States Department of Energy and Alliance for Sustainable Energy, LLC, the Manager and Operator of the National Renewable Energy Laboratory.

### BACKGROUND

Industrially, furfural has been used for resin production and as a solvent for lubricant production. More recently, furfural has been reported to produce furan-based chemicals such as methylfuran, tetrahydro furfuryl alcohol, and furfuryl alcohol which have potential for industrial applications. HMF is identified as one of the top value-added chemicals that can be obtained from biomass and is an important intermediate for the synthesis of useful chemical building blocks such as 2,5-dimethylfuran, 2,5-diformylfuran, and 2,5-furandicarboxylic acid (2,5-FDCA). Considering the importance of furfural and HMF as valuable chemical intermediates, the production of furfural on an industrial scale via acid catalyzed dehydration of pentosan-rich agricultural residues dates back to as early as 1920s. Though thermocatalytic conversion of sugars to furfurals (furfural and HMF) has been widely studied, however, owing to the industrial relevance and importance of these valuable intermediates, quest for advanced approaches which can allow to further improve their molar yields are highly desirable.

Both furfural and HMF are known to be obtained from pentoses and hexoses, respectively in an acid catalyzed reaction using homogeneous and heterogenous acid catalysts. The most widely employed homogenous catalysts to produce furfurals are mineral acid catalysts with primarily hydrochloric and sulfuric acids being the most commonly used for the production of furfurals via batch operations. Others have reported the dehydration of glucose and xylose to HMF and furfural, respectively in yields approaching 23% and 65% using HCl as a catalyst and MIBK: 2-butanol (7:3) as organic solvent and water: DMSO (5:5) as the aqueous phase. Others have reported the yields of 86% furfural and 21% HMF obtained from dehydration of sugars in maple wood using 1 wt % sulfuric acid and tetrahydrofuran as a cosolvent. As shown in these and other works, the extraction of furfurals in organic solvents result in enhanced furfurals yield. However, dehydration of aldose sugars using homogenous Brønsted acid catalysts often result in low product yields and selectivity as in addition to dehydration a variety of side reactions such as condensation, fragmentation and rehydration reactions also occur.

Other objects, advantages, and novel features of the present invention will become apparent from the following detailed description of the invention when considered in conjunction with the accompanying drawings.

### SUMMARY

In an aspect, disclosed herein is a method for the simultaneous dehydration of xylose and glucose in biomass hydrolysate to furfurals comprising the step of contacting the biomass hydrolysate with solid acid catalysts; and further comprising the step of contacting the biomass hydrolysate with a silica-alumina catalyst in the presence of a catalytic amount of NaCl; and further comprising contacting the biomass hydrolysate with an acidic ion-exchange resin in a homogeneous solvent system of aqueous dioxane. In an embodiment, the solid acid catalyst is selected from the group consisting of acidic ion-exchange resins, metal oxides, zeolites and amorphous silica-alumina catalysts catalyst. In an embodiment, the silica-alumina catalyst is Davicat 3115. In an embodiment, the yield of furfurals from the biomass hydrolysate is about 96% or greater. In an embodiment, the yield of furfural is 5-hydroxymethylfurfural and is greater than about 74%. In an embodiment, the yield of combined furfurals is greater than about 80%. In an embodiment, the catalytic amount of NaCl is about 33 mM. In an embodiment, the acidic ion-exchange resin is Purolite 275-CT. In an embodiment, the aqueous dioxane solvent system consists of a mixture of 2 parts dioxane to 1 part water (vol/vol). In an embodiment, the dehydration reaction is at about 197 degrees Celsius. In an embodiment, the dehydration reaction is for about 5 minutes. In an embodiment, the dehydration reaction is in a microwave reactor.

In an aspect, disclosed herein is a method for simultaneous dehydration of glucose and xylose present in a biomass hydrolysate to furfural and 5-hydroxymethylfurfural using a heterogenous solid acid catalyst in a microwave reactor. In an embodiment, the solid acid catalyst is selected from the group consisting of acidic ion-exchange resins, metal oxides, zeolites and amorphous silica-alumina catalysts catalyst. In an embodiment, the dehydration is at about 197 degrees Celsius. In an embodiment, the dehydration reaction is for about 5 minutes. In an embodiment, the biomass hydrolysate is corn stover hydrolysate. In an embodiment, the yield of furfural is 96%. In an embodiment, the yield of furfural is 5-hydroxymethylfurfural and is greater than about 74%. In an embodiment, the yield of furfurals is greater than about 80%.

Other objects, advantages, and novel features of the present invention will become apparent from the following detailed description of the invention when considered in conjunction with the accompanying drawings.

### BRIEF DESCRIPTION OF THE DRAWINGS

FIGS. 1A and 1B depict glucose conversion (FIG. 1A) and HMF and LA yields (FIG. 1B) from dehydration of 8 wt % glucose at 195° C. /5 min using dioxane: water— 2:1 (v/v) with different homogeneous and heterogenous catalysts.

FIGS. 2A and 2B depict glucose conversion (FIG. 2A) and HMF and LA yields (FIG. 2B) from dehydration of 8 wt % glucose at 195° C. /5 min using dioxane: water— 2:1 (v/v) with the addition of Pur-275 to various solid catalysts.

FIGS. 3A and 3B depict the effect of NaCl concentration on glucose conversion (FIG. 3A) and HMF and LA yields (FIG. 3B) from dehydration of 8 wt % glucose at 195° C. /5 min using dioxane: water— 2:1 (v/v) with ASA-15 and Pur-275 as solid catalysts.depicts.

FIGS. 4A and 4B depict glucose conversion (FIG. 4A) and HMF yields (FIG. 4B) from dehydration of 8 wt % glucose at 195° C./5 min dioxane: water— 2:1 (v/v) with the addition of NaCl at 33 mM and Pur-275 to various solid catalysts.

FIGS. 5A and 5B depict glucose conversion (FIG. 5A) and HMF yields (FIG. 5B) from dehydration of 8 wt % glucose at 195° C. /5 min using dioxane: water— 2:1 (v/v) with and without the addition of NaCl at 33 mM with recycling ASA-15 and Pur-275 for five consecutive runs.

FIGS. 6A and 6B depict xylose conversion (FIG. 6A) and Furfural yields (FIG. 6B) from dehydration of 6 wt % xylose at 195° C. /5 min using dioxane: water— 2:1 (v/v) with the addition of 33 mM NaCl and Pur-275 to various solid catalysts.

FIGS. 7A and 7B depict glucose and xylose conversion (FIG. 7A) and HMF and furfural yields (FIG. 7B) from dehydration of DMR hydrolysate (9% glucose and 4% xylose) at 195° C./5 min using dioxane: water— 2:1 (v/v) with the addition of 33 mM NaCl to ASA-15 and Pur-275.

FIGS. 8A, 8B, 8C depict (FIG. 8A) Glucose 8 wt % and fructose 8 wt % conversion, (FIG. 8B) HMF and LA yields from dehydration of glucose and fructose, (FIG. 8C) HMF yields with dehydration of 4 wt % HMF at 195° C./5 min using dioxane: water— 2:1 (v/v) with or without the addition of 33 mM NaCl to ASA-15 and Pur-275 added individually or together.

FIG. 9, depicts HPLC chromatograms showing isomerization of glucose to fructose after dehydration of 8 wt % glucose at 195° C./5 min using dioxane: water— 2:1 (v/v) with different heterogenous catalysts.

FIGS. 10A, 10B, depicts glucose conversion (FIG. 10A) and HMF and LA yields (FIG. 10B) from dehydration of 8 wt % glucose as a function of reaction temperature for a reaction period of 5 min using dioxane: water— 2:1 (v/v) with ASA-15 and Pur-275 as solid catalysts and 33 mM NaCl.

FIGS. 11A, 11B, depicts glucose conversion (FIG. 11A) and HMF yields (FIG. 11B) from dehydration of 8 wt % glucose as a function of ASA-15 and Pur-275 loadings. Dehydration were performed at 195° C./5 min using dioxane: water— 2:1 (v/v) with the addition of NaCl at 33 mM.

### DETAILED DESCRIPTION

Disclosed herein are methods and compositions of matter for simultaneous dehydration of glucose and xylose present in a process-relevant biorefinery hydrolysate to furfural and 5-hydroxymethylfurfural (HMF) using heterogenous solid acid catalysts in a microwave reactor. Initially, several solid acid catalysts with varied Brønsted and Lewis acidity were screened to evaluate their activity and selectivity in dehydration of pure glucose to HMF. A noticeable improvement in HMF yield from dehydration of 8 wt % glucose was obtained by combining an acidic ion-exchange resin (Purolite CT-275) with an amorphous silica-alumina catalyst (ASA-15) resulting in HMF yields of 27-33% using a homogeneous solvent system of aqueous dioxane (dioxane: water— 2:1 v/v) at 195° C. and 5 minutes. Under the same reaction conditions, catalysts, and solvent system but with the addition of NaCl in catalytic amounts (33-100 mM), a more than two-fold increase in HMF yields (66-70%) was achieved for the dehydration of 8 wt % glucose whereas furfural yields approaching 95% were achieved for the dehydration of 6 wt % xylose when conducted separately. Notably, using the same catalyst and solvent system while slightly modifying the reaction conditions to 197° C., 5 minutes, simultaneous dehydration of 4 wt % xylose and 9 wt % glucose present in a process-relevant corn stover hydrolysate resulted in furfural and HMF yields of 96% and 74%, respectively resulting in a combined furfurals yield of 80%. The results further show that pH of the reaction solution plays an important role where the addition of solid catalysts and NaCl altered the pH and influenced the selectivity of the products and by-products. The pH<2 resulted in low HMF yields due to the increased formation of HMF degradation products while a pH 2-3 resulted in attaining high HMF yields by possibly stabilizing the reaction intermediates and product and suppressing the side reactions.

In an embodiment, the following are the advantages of using this approach (1) Replacement of one-time use expensive homogenous acid catalysts with recyclable heterogenous solid acid catalysts in simultaneous dehydration of mixed sugars present in biomass hydrolysate to furfurals (2) No requirement for separate chemical or bio—catalytic isomerization of the aldoses (glucose and xylose) into their respective ketoses (fructose and xylulose) to achieve their efficient dehydration to HMF and furfural (3) Simultaneous dehydration of all sugars in a biomass hydrolysate to furfurals in unprecedented high yields, i.e., 96% furfural and 74% HMF resulting in a combined furfurals yield of 80%.

5-(Hydroxymethyl) furfural (HMF) and furfural, which are identified as important precursors to renewable fuels and chemicals, can be produced via respective dehydration of glucose and xylose. Despite the significant amount of work that already exists on dehydration of pentoses and hexoses to furfural and HMF, respectively, most of the previous works have investigated the dehydration of single sugars such as xylose or fructose/glucose with a very limited amount of work being reported on the dehydration of mixed sugars simultaneously. As the biorefinery concept receives increased attention, where renewable lignocellulosic biomass is envisioned to be used as a feedstock, this will require the utilization of biomass derived hydrolysates that are comprised of mixed sugars, i.e., both hexoses and pentoses. However, the reaction conditions needed for efficient dehydration of pentoses and hexoses are very different with hexoses generally requiring more severe reaction conditions. Therefore, efficient production of furfurals from biorefinery hydrolysates pose a significant challenge as the reaction conditions required for dehydration of each sugar is considerably different. In addition, the presence of impurities in biorefinery hydrolysates such as soluble lignin may result in increased side reactions and catalyst deactivation affecting furfurals yield requiring further optimization of reaction conditions to obtain high furfurals yield from biorefinery hydrolysates.

As disclosed herein, various solid acid catalysts such as acidic ion-exchange resins, metal oxides, zeolites and amorphous silica-alumina catalysts were evaluated for dehydration of mixed sugars present in biomass (DMR) hydrolysate to furfurals. Dehydration of xylose and glucose present in biomass hydrolysate to furfurals in unprecedented high yields, i.e., 96% furfural and 74% HMF yields resulting in a combined furfurals yield of 80% was demonstrated using an amorphous silica-alumina catalyst (Davicat 3115, W. R. Grace & Co.) in the presence of a catalytic amount of NaCl (33 mM) and an acidic ion-exchange resin (Purolite 275-CT) in a homogeneous solvent system of aqueous dioxane (dioxane:water— 2:1 v/v) at 197° C. for 5 min in a microwave reactor. This approach eliminated the need for separate chemical or bio-catalytic isomerization of the aldoses (glucose and xylose) into their respective ketoses (fructose and xylulose) to achieve their efficient dehydration to HMF and furfural and presents a potential scenario of significant cost savings in the production of these fuel precursors directly from biomass hydrolysates.

Thus, in an embodiment, disclosed herein is a system that allows for a much higher level of isolation of the furfural than using prior systems; the combination of known solid acid and base catalysts with low level amount of sodium chloride produces an unexpected amount of furfurals in the organic layer.

A furfural and 5-hydroxymethylfurfural (HMF) are identified as valuable chemical intermediates in the production of biobased chemicals and biofuels.

Previously, high yields of HMF and furfural have been reported from ketose sugars relative to glucose due to slow enolization of the aldose sugars which is identified as a rate limiting step in the dehydration of aldose sugars. Noticeable improvement in furfural and HMF yields from aldose sugars have been reported by combining Lewis and Brønsted acid catalysts where the Lewis and Brønsted acids catalyze the isomerization of aldose to ketose form and dehydration of ketoses to furfurals, respectively resulting in high furfurals yield (Scheme 1).

Scheme 1 depicts a reaction scheme for efficient dehydration of glucose to HMF by combining Lewis and Brønsted acid catalysts.

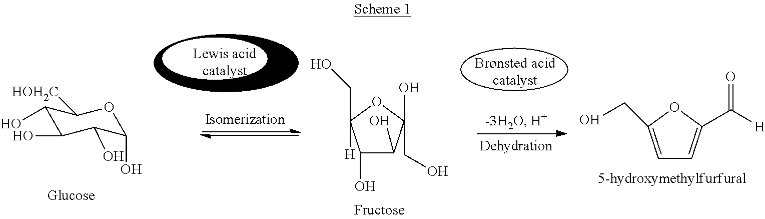

In addition to combining the Lewis and Brønsted acid catalysts, addition of metal halides, especially NaCl, has also been investigated by several researchers showing that addition of a salt resulted in enhanced extraction of furfurals into the organic solvent which is attributed to salting-out effect of the salt. Although, the main objective of salt addition in most of the previous studies was reported to achieve enhanced extraction of furfurals into the organic solvent while some studies have also reported the effect of salts in promoting the rate of dehydration of sugars to furfurals. For example, others have reported that the Cl— ions promoted the formation of the 1,2-enediol from the acyclic form of xylose which is considered to be the rate limiting step in the formation of furfural from aldose sugars.

More recently, it has been reported that a ten-fold increase in reactivity in the conversion of fructose to HMF with the addition of catalytic amounts of NaCl (e.g., 5 mM) and 5 mM HCl can occur. They showed with the molecular dynamics simulations that for acid-catalyzed dehydration of fructose, chloride ions stabilize protonated transition states of the cationic intermediates resulting in high yield and selectivity of HMF. Despite of obtaining high furfurals yield using homogenous acid catalysts, the problems such as reactor corrosion and limitations in catalyst separation and recycling limits their use for industrial applications thus necessitating their replacement with heterogenous solid catalysts.

The use of heterogenous acid catalysts in the production of furfurals is useful due to their ease of handling, separation, recovery and regeneration. In dehydration of hexoses others have reported 54.1 and 37.2% HMF yields from fructose and glucose, respectively with mesoporous TiO₂ using DMSO/water as the solvent system in 5 min reaction at 120° C. Others have reported 81 and 45% HMF yields at glucose concentrations of 2 and 10 wt %, respectively after 3 h reaction at 175° C. with phosphated TiO₂ in a water—butanol biphasic solvent system. In another study, the same group reported 86% HMF yield at a glucose concentration of 5 wt % using a catalyst mixture of TiO₂—ZrO₂/Amberlyst 70 and 4 wt % NaCl using dioxane/water (4/1) solvent system after 3 h reaction at 175° C. Others have reported a high HMF yield approaching 86% at a glucose concentration of 6 wt % using a combination of polymer bound sulfonic acids (PEG-OSO₃H/PS-PEG-OSO₃H) and LiCl using DMSO/water (2/1) solvent system in 1.5 h reaction at 120° C. Others have attributed high HMF yields to a combined action of Lewis and Brønsted acid catalysts in achieving efficient isomerization and dehydration reactions in a one-pot reaction system. In dehydration of xylose, furfural yield of up to 50% was reported with sulfated zirconia at 160° C. for 4 h in water/toluene (3/7) as a solvent. Others have obtained 72% furfural yields with micro-mesoporous sulfonic acid (MCM-41) at 140° C. in 24 h using DMSO or water/toluene as a solvent. Others have reported 81% furfural yield at a xylose concentration of 2 wt % after 2 h reaction at 175° C. with mordenite in a γ-Valerolactone/water (9/1) solvent system.

Despite of significant amount of work that already exists on dehydration of pentoses and hexoses to furfural and HMF, respectively, most of the previous works have investigated the dehydration of single sugars such as xylose or fructose/glucose with a very limited amount of work being reported on the simultaneous dehydration of mixed sugars especially biorefinery hydrolysates. As the biorefinery concept receives increased attention, where renewable lignocellulosic biomass is envisioned to be utilized as a feedstock for the production of biofuels, chemicals and bioproducts, the utilization of biomass derived hydrolysates that are comprised of both hexoses and pentoses will be essential. However, the reaction conditions needed for efficient dehydration of pentoses and hexoses are very different with hexoses generally requiring more severe reaction conditions. Therefore, efficient production of furfurals from biorefinery hydrolysates pose a significant challenge as the reaction conditions required for dehydration of each sugar is different. In addition, the presence of impurities in biorefinery hydrolysates such as soluble lignin may result in increased side reactions and catalyst deactivation affecting furfurals yield requiring further optimization of reaction conditions to obtain high furfurals yield from biorefinery hydrolysates.

In that regard, the main objective of this work is to maximize furfurals production using heterogenous solid acid catalysts via simultaneous dehydration of pentoses and hexoses present in a process-relevant biorefinery hydrolysate obtained from corn stover using deacetylation and mechanical refining (DMR) process. Various solid acid catalysts such as acidic ion-exchange resins, metal oxides, zeolites and amorphous silica-alumina (ASA) catalysts are evaluated and are compared with two homogenous catalysts e.g., AlCl₃.6H₂O and HCl. First, the optimization of reaction variables including reaction time, temperature, catalyst type, and catalyst loading was achieved for dehydration of 8 wt % glucose to HMF. These reaction conditions were then implemented on dehydration of 6 wt % xylose to furfural which were then fine-tuned to maximize the production of furfurals from simultaneous dehydration of glucose and xylose present in a process-relevant biorefinery hydrolysate. In addition to evaluating the activity, selectivity and stability of the solid catalysts their recyclability was also evaluated.

Results and Discussion

Solid Catalysts Screening

Various solid acid catalysts such as acidic ion-exchange resins (Purolite CT-275 (Pur-275), and Nafion NR-50 (Naf-50)), metal oxides (TiO₂, ZrO₂, Nb₂O₅, and WO₃, and TiO₂— SiO₂), zeolites (H-ZSM-5, and H-BEA), and amorphous silica-alumina catalysts (ASA) (Davicat 3115 (ASA-15) and Davicat 3125 (ASA-25) are evaluated. Physico-chemical properties of solid catalysts used in this work are provided in Table 1.

TABLE 1
Physico-chemical properties of solid catalysts

| | Surface area | Total acid sites | B sites | L sites |
|---|---|---|---|---|
| Catalyst | (m²/g) | (u mol/g) | (u mol/g) | (u mol/g) |
| Pur-275 | 20-40 | | | |
| Naf-50 | | | | |
| TiO₂ | 47 | 83 | — | 83 |
| ZrO₂ | 73 | 102 | | |
| Nb₂O₅ | 54 | 58 | | |
| WO₃ | 12 | 30 | 4 | 26 |
| H-ZSM5 | 430 | 1250 | 1110 | 140 |
| H-BEA | 650 | 1280 | 1090 | 190 |
| TiO₂—SiO₂ | 53 | 320 | — | 320 |
| ASA-15 | 370 | 980 | 470 | 510 |
| ASA-25 | 480 | 1100 | 400 | 700 |

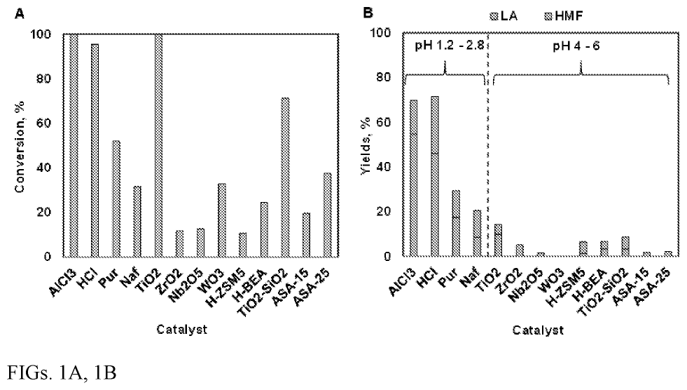

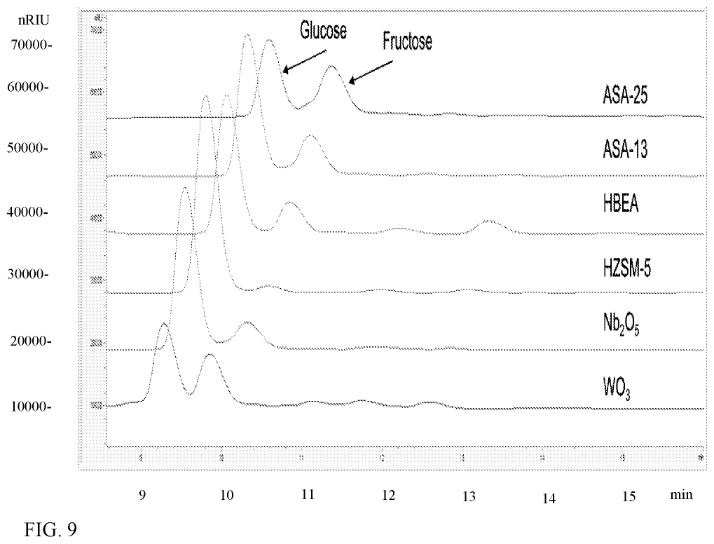

FIG. 1 shows the conversion of glucose and HMF yield for the experiments where various solid acid catalysts were screened to assess their activity in dehydration of glucose to HMF. Experiments were conducted with 8 wt % glucose at 195° C. for 5 min in a homogeneous solvent system of aqueous dioxane (dioxane: water— 2:1 v/v) including control experiments with two homogenous catalysts, e.g., AlCl₃ and HCl which were added individually at the concentrations of 10 and 25 mM, respectively. With the homogenous catalysts, AlCl₃ and HCl, HMF yields of 60 and 55%, respectively were obtained whereas with all the solid catalysts tested, the maximum HMF yield of 17.5% with Pur-275 was obtained. For the experiments conducted with solid catalysts, the HPLC chromatograms of the reaction solutions obtained after dehydration reaction showed the presence of fructose in varying concentrations for different catalysts confirming that the isomerization of glucose to fructose occurred, however, hardly any dehydration of glucose or fructose took place suggesting the presence of sufficient active Lewis acid sites but not enough Brønsted acid sites on the solid catalysts tested. Among the various catalysts screened, ASA-25 and WO₃ showed the highest conversion to fructose (FIG. 9). The pH of hydrolysates measured post dehydration reaction was found to be greater than 4 for all the solid catalysts except for acidic ion-exchange resins, i.e., Naf-50 and Pur-275 for which a pH of 2.8 was obtained with each catalyst. In contrast, for the experiments conducted with the homogeneous catalysts (AlCl₃ or HCl), pH was found to be in between 1.2-2.0. Though in this pH range high HMF yields were obtained but it also resulted in the generation of HMF degradation products in significant yields (levulinic acid (LA)— 15-25%) (FIG. 1B).

Recently, in the acid catalyzed dehydration of glucose Guo et. al. have reported high HMF yield (53%) accompanied with increased production of LA and formic acid (FA) in the pH range of 1-2 with a reduction in LA and FA production in the pH range of 2-4. These results suggest that a pH between 2.0-4.0 may assist in achieving efficient dehydration of glucose to HMF while preventing the formation of HMF degradation products. Additionally, others have reported that a combination of catalysts with both Lewis and Brønsted acidity was required to obtain dehydration of glucose to HMF in high yields. Our results also suggest that for solid acid catalysts where the pH was above 4, the addition of a solid catalyst with Brønsted acidity would be required to attain the pH<4 and maintain it between 2-4 to achieve an efficient dehydration of glucose to HMF.

Effect of Addition of Purolite CT-275

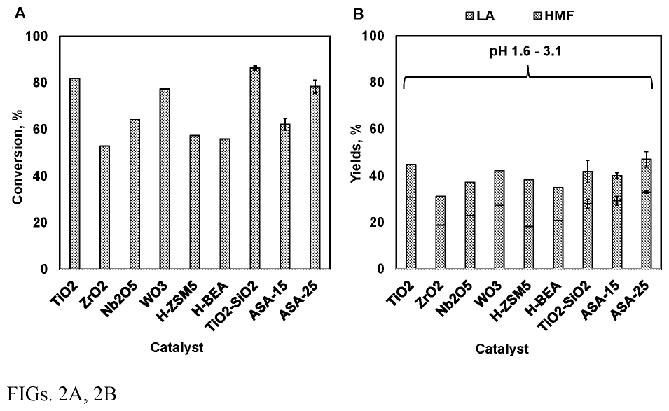

Next, glucose dehydration experiments were conducted by combining Pur-275 to all the solid catalysts tested above to attain a pH value between 2-4 which should allow both reactions, i.e., isomerization of glucose to fructose and dehydration of both glucose and fructose to HMF to occur. Pur-275 was selected over Naf-50 as a Brønsted acid catalyst as it showed better HMF yields (FIG. 1). Experiments were conducted with 8 wt % glucose at 195° C. for 5 min using dioxane to an aqueous ratio of 2:1. The conversion of glucose and HMF yields are shown in FIGS. 2A and 2B, respectively. As can be seen in FIG. 2A, barring TiO₂, a significant increase in glucose conversion ranging from 52-78% was obtained for all solid catalysts with the addition of Pur-275 when compared to low glucose conversion of 12-37% obtained without its addition (FIG. 1A).

In line with glucose conversion, a significant increase in HMF yield was observed with the addition of Pur-275 to all the solid catalysts (FIG. 2B). For instance, a three-fold increase in HMF yield from 10 to 30% was obtained for TiO₂. For other metal oxides and zeolites where a less than 5% HMF yield was obtained without the addition of Pur-275, HMF yield ranging between 18-27% was obtained with the addition of Pur-275. For amorphous silica-alumina catalysts (ASA-15 and ASA-25), a remarkable fifteen-fold increase in HMF yield from less than 2% to 33% was obtained with the addition of Pur-275. Interestingly, production of LA in yields approaching 10-20% was also observed with the highest being obtained for H-ZSMS (20%) and lowest for ASA-15 (10%). The addition of Pur-275 resulted in attaining the pH between 1.6-3.1 which certainly helped in increased dehydration of glucose/fructose to HMF but also resulted in increased LA yields. The high yields of LA suggest that a further optimization of reaction conditions (i.e., increasing the reaction temperature and decreasing reaction time) or reduction in Pur-275 loading may result in enhancing HMF yields by minimizing the production of LA.

Effect of NaCl Addition

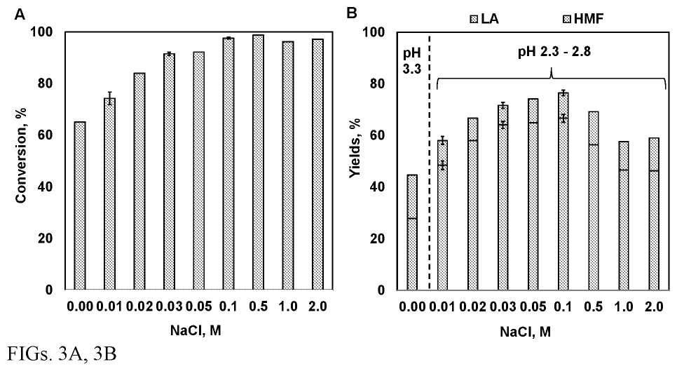

Several researchers have studied the role of metal chlorides, especially NaCl, in acid catalyzed dehydration of aldose sugars to furfurals. These studies showed that the presence of chloride ions, even in catalytic amounts, promote the formation of 1, 2-enediol and the stabilization of critical cationic intermediates from aldose sugars thus resulting in increased sugar conversion and enhanced furfurals yield. In addition, NaCl is also reported to reduce the pH of the aqueous solution by providing H⁺ ions thereby acting as a catalyst in dehydration of sugars under acidic conditions.42 As such, dehydration experiments were conducted with 8 wt % glucose upon adding NaCl at varying concentrations (0-2 M) while using a combination of ASA-15 and Pur-275 as Lewis and Brønsted solid acid catalysts, respectively. ASA-15 was selected over other Lewis acid catalysts as among all the Lewis catalysts tested in this work higher selectivity and better mass closure was achieved with ASA-15 (data not shown). FIGS. 3A and 3B show the conversion of glucose and HMF yield, respectively. As can be noted in FIG. 3A, a glucose conversion of 65% was obtained when no NaCl was added which steadily increased to 98% as the NaCl concentration was increased to 100 mM under the same reaction conditions (195° C., 5 min). On further increasing the NaCl concentration to 500 mM only a modest increase in glucose conversion was observed which slightly decreased on further increasing the NaCl concentration to 2 M. In contrast, a significant increase in HMF yield was obtained with the addition of NaCl in catalytic amounts (10-100 mM) when compared to the HMF yield obtained without NaCl addition (FIG. 2B). A more than two-fold increase in HMF yield (58%) was obtained with the addition of 20 mM NaCl which increased to 68% upon increasing the NaCl concentration to 100 mM. The significant increase in HMF yield from dehydration of aldohexose obtained with the addition of NaCl even in catalytic amounts supports the previously reported mechanism where the presence of Cl— favors the formation of the 1,2-enediol from aldose sugar which readily reacts to form HMF under acidic conditions. However, on further increasing the NaCl concentration above 100 mM resulted in a slight drop in HMF yield. This suggests that up to a certain concentration of chloride ions, optimum reaction conditions exist that result in enhancing the HMF yield, but upon increasing NaCl beyond a certain threshold concentration results in increased yield loss reactions possibly due to an increase in the concentration of reactive intermediates and their side reactions.

For the experiments performed with NaCl addition, the measurement of the solution pH at room temperature post-dehydration reaction revealed a decrease in pH from 3-4 to 2-3. This suggests that in the presence of NaCl, Na+ undergo the ion exchange reaction on the acid sites of the acidic ion-exchange resin catalyst (Pur-275) thus releasing H+ ions which aid in both decreasing the pH and increasing the Brønsted acidity of the solution resulting in efficient dehydration of glucose to HMF.

Effect of Reaction Time and Temperature

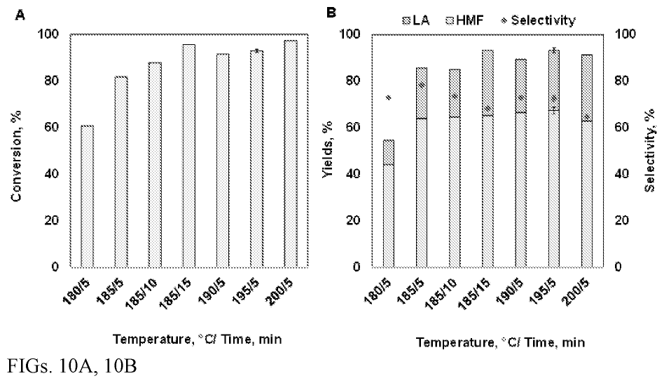

In our previous work on direct dehydration of biomass sugars using homogenous catalysts (AlCl₃ and HCl) revealed that high furfurals yields were obtained by implementing short residence times (3-5 min at 200° C.). Therefore, to find the optimum reaction conditions to maximize HMF yields from glucose using heterogenous catalysts, experiments were conducted in this work by varying reaction temperature and time while using the same combination of catalysts, i.e., ASA-15 and Pur-275 and NaCl at 33 mM and using dioxane: water— 2:1 (v/v). The conversion of glucose and HMF yields are shown in FIGS. 10A and 10B, respectively. It can be noted from FIG. 10, at 180° C. and 5 min the glucose conversion, HMF yield and selectivity of 60%, 44% and 72%, respectively were obtained which increased to 82%, 64%, and 78%, respectively at 185° C. and 5 min. Interestingly, keeping the reaction temperature constant at 185° C. but increasing the reaction period to 15 min, the glucose conversion increased to 96%, however, no noticeable increase in HMF yield was obtained possibly as a result of an increase in rehydration of HMF to LA which increased from 21 to 28% resulting in reduction in HMF selectivity from 78 to 68%. On increasing the reaction temperature to 195° C. while keeping the reaction time at 5 min, both the glucose conversion and HMF yield increased steadily to 93 and 68%, respectively. On further increasing the reaction temperature to 200° C., a slight increase in glucose conversion to 97% was obtained however a slight drop in HMF yield to 63% was also obtained likely due to an increase in both rehydration of HMF to LA and degradation reactions. From the above experiments, for dehydration of 8% glucose the optimum reaction conditions at which the maximum HMF yield of 68% was obtained were found to be 195° C. and 5 min using ASA-15 and Pur-275 and NaCl at 33 mM with dioxane to an aqueous ratio of about 2:1.

Effect of NaCl Addition on Different Solid Catalysts

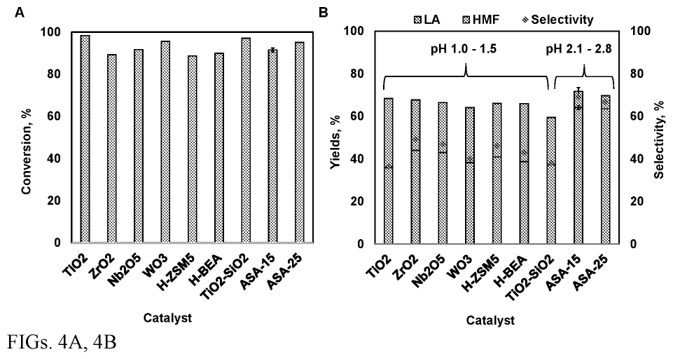

Next, to select the best Lewis and Brønsted acid catalyst combination for dehydration of glucose to HMF, all the catalysts were reevaluated in the presence of 33 mM NaCl while combining each solid catalyst with Pur-275. Experiments were conducted with 8 wt % glucose under the optimum reaction conditions obtained above. The conversion of glucose and HMF yields are shown in FIGS. 4A and 4B, respectively. As can be seen in FIG. 4A, a significant increase in glucose conversion was obtained for all solid catalysts with more than 90% conversion being obtained with the addition of 33 mM NaCl whereas only 52-78% conversion obtained without the addition of NaCl (FIG. 2A). Similar to glucose conversion, a significant increase in HMF yield was obtained with the addition of 33 mM NaCl to solid catalysts especially with ASA-15 and ASA-25. For various metal oxides and zeolites tested, except for TiO₂ and TiO₂—SiO₂, minimally a 50% increase in HMF yield was obtained with all other catalysts with the addition of 33 mM NaCl, when compared to HMF yields obtained without the addition of NaCl (FIG. 2B). For both amorphous silica-alumina catalysts tested (ASA-15 and ASA-25) a more than two-fold increase in HMF yields approaching 65-68% was obtained with the addition of 33 mM NaCl when compared to HMF yields obtained without the addition of NaCl (FIG. 2B). Interestingly, an increase in the generation of LA was also observed with all the catalysts tested except for ASA-15 and ASA-25 for which a slight decrease in LA yields was observed. Moreover, the pH of the reaction solution was found to be around 1.0-1.5 for all the catalysts tested whereas it was found to be between 2.1-2.8 for ASA-15 and ASA-25. As can be noted from Table 1, zeolites provide much stronger Brønsted acidity, and therefore, is not surprising to see lower pH for the metal oxides and zeolites as compared to ASA. Overall, the results clearly show that the pH of the reaction solution plays an important role in dehydration of sugars to furfurals where a pH<2 results in low HMF yields due to the formation of degradation products in high yields, while a pH between 2-3 facilitates in attaining high HMF yields by suppressing the degradation reactions.

Effect of Catalyst Loading

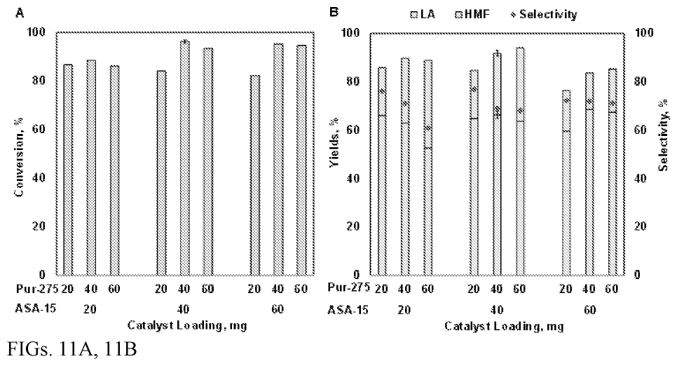

In the previous experiments, to find the optimum reaction conditions, each catalyst (ASA-15 and Pur-275) was added at about 40 mg/57 mg of glucose. To investigate the influence of catalyst loading on glucose conversion and HMF yield, both catalysts were varied at three different loadings by increasing the loading for one catalyst while keeping the other catalyst constant. Experiments were conducted with 8 wt % glucose under the optimum reaction conditions obtained above with the addition of 33 mM NaCl. The effect of increased loadings of either Lewis or Brønsted acid catalysts on glucose conversion and HMF yields is shown in FIG. 11. It can be noted in FIG. 11, on keeping the Lewis acid catalyst (ASA-15) constant at 20 mg and increasing the Brønsted acid catalyst (Pur-275) from 20 to 60 mg/57 mg glucose, glucose conversion remained constant at 86±1% whereas HMF yield and selectivity decreased from 66 to 52% and 76 to 61%, respectively. The reduction in HMF yield can be attributed to increased Brønsted acidity resulting in increased rehydration of HMF to LA which is evident from the increased yield of LA from 20 to 36%. Increasing the loading of ASA-15 to 40 mg and increasing the Brønsted acid catalyst (Pur-275) from 20 to 60 mg/57 mg glucose, glucose conversion increased from 84 to 96% whereas HMF yield remained constant at 66% and then decreased slightly to 64% possibly due to increased degradation reactions at increased Brønsted acidity. Finally, on increasing the addition of ASA-15 to 60 mg and then increasing the Brønsted acid catalyst (Pur-275) from 20 to 60 mg/57 mg glucose, glucose conversion increased from 82 to 95% and HMF yield increased from 60 to 68%. For the addition of ASA-15 at highest loading, though lower yields of LA (17%) were obtained but this catalyst loading also resulted in low HMF selectivity of 72% suggesting no significant benefit of increasing ASA-15 loading to 60 mg/57 mg of glucose. The results of this experiment show that high HMF yield and selectivity was obtained at low to moderate loadings (20 to 40 mg) of both catalysts, i.e., ASA-15 and Pur-275 with no significant benefit observed for adding catalysts at high loadings.

Catalyst Recycling

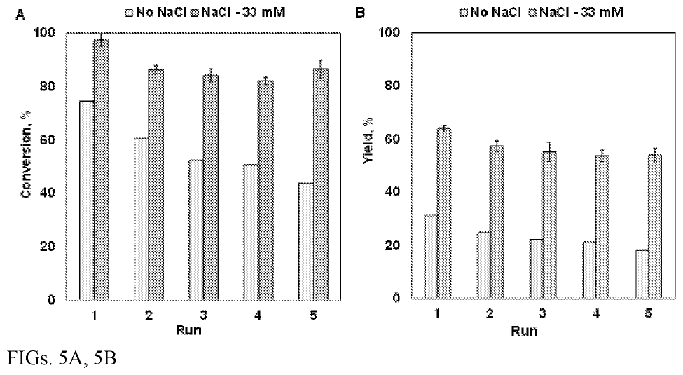

The main advantages of using heterogenous catalysts are their ease of handling, separation, recovery, and reusability. As, such reusability of the best combination of solid catalysts (ASA-15 and Pur-275) for glucose dehydration reactions was evaluated. The reusability of the solid catalysts was examined for five consecutive runs with the addition of fresh solvent and substrate after each run without implementing any catalyst regeneration or washing step in between the runs. Dehydration experiments were conducted with 8% glucose at 195° C. and 5 min using ASA-15 and Pur-275 without and with the addition of 33 mM NaCl to evaluate the influence of salt addition on the reusability of catalysts. The conversion of glucose and HMF yields are provided in FIGS. 5a and 5b, respectively. The results show that the conversion of glucose and HMF yield obtained between the first and fifth runs decreased from 75 to 44% and 31 to 18%, respectively when no salt was added. With the addition of 33 mM NaCl, as expected a more than two-fold increase in HMF yields was obtained for each run, however, a decrease in the conversion of glucose and HMF yield still persisted which decreased from 96 to 85% and 65 to 55%, respectively. Clearly, regardless of the NaCl addition a decrease in HMF yield was observed albeit for only first three runs with the salt addition but was more noticeable for the runs without salt addition. The measurement of pH for the experiments performed without and with 33 mM NaCl addition revealed an increase in pH from 3 to 4 and 2 to 3, respectively from run #1 to run #5 suggesting a partial loss in the activity of the catalysts. Since no washing or catalyst regeneration step was included between the catalyst recycle runs, implementing these steps after each recycle run may compensate for the loss in catalysts activity and result in maintaining constant glucose conversion and HMF yield.

Dehydration of Xylose to Furfural

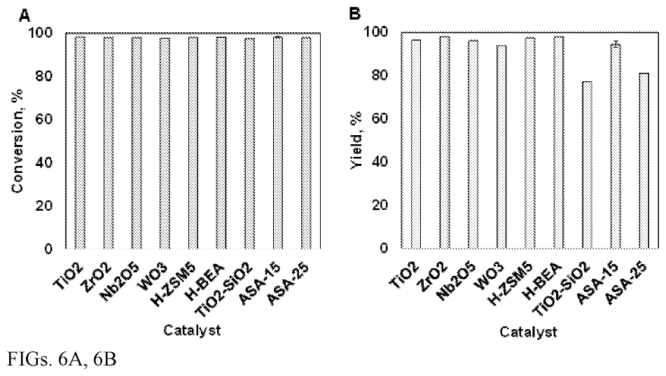

Next, dehydration of 6 wt % xylose to furfural was evaluated by implementing the optimum reaction conditions obtained for dehydration of glucose above. Similar to dehydration of glucose experiments, to select the best Lewis and Brønsted acid catalyst combination for dehydration of xylose to furfural all the catalysts were evaluated in the presence of 33 mM NaCl while combining each catalyst with Pur-275. The conversion of xylose and furfural yields are shown in FIGS. 6A and 6B, respectively.

As can be seen in FIG. 6A, a high xylose conversion of more than 97% was obtained for all solid catalysts. Correspondingly, high furfural yields greater than 95% were obtained with all the solid catalysts except for TiO₂-SiO2 and ASA-25 for which lower furfural yields of 77 and 81%, respectively were obtained (FIG. 6B). In dehydration of glucose, among all the solid catalyst tested highest HMF yields were obtained with both amorphous silica-alumina catalysts (ASA-15 and ASA-25). However, in dehydration of xylose highest furfural yields were obtained with zeolites (98% with H-BEA) while a slightly lower furfural yield of 95% was obtained with metal oxides and ASA-15 and a much lower furfural yield of 81% was obtained with TiO₂— SiO₂ and ASA-25. These results suggest that the solid catalysts with high Brønsted acidity result in high furfural yields.

Simultaneous dehydration of xylose and glucose present in a process-relevant biorefinery hydrolysate to furfurals.

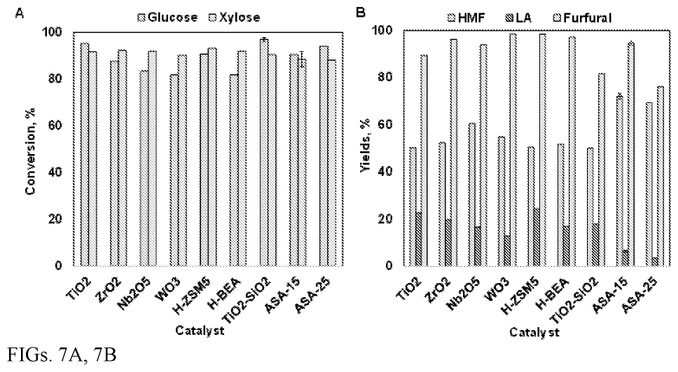

Initially, simultaneous dehydration of xylose and glucose present in DMR hydrolysate to furfurals was evaluated by implementing the optimum reaction conditions obtained for glucose dehydration. The glucose and pentose concentrations in the hydrolysate were determined to be 9 wt % and 4 wt %, respectively with about 7% of the sugars in the hydrolysate present as oligosaccharides. To select the best Lewis and Brønsted acid catalyst combination for dehydration of sugars in DMR hydrolysate, all the catalysts were evaluated in the presence of 33 mM NaCl while combining each catalyst with Pur-275. The conversions of glucose and xylose, and furfural, HMF and LA yields are shown in FIGS. 7A and 7B, respectively.

As can be seen in FIG. 7A, for various solid catalysts tested a glucose conversion ranging from 82% to 97% was obtained with the highest glucose conversion of 95 and 97% were being obtained with TiO₂ and TiO₂-SiO2, respectively. Interestingly, despite obtaining highest glucose conversion, HMF yields obtained with these two catalysts were on the lower end of the range (51-72%) of HMF yield being obtained with all the catalysts (FIG. 7B). Similar to the results of HMF yields obtained with pure glucose, the highest HMF yield approaching 72% was obtained with ASA-15 from dehydration of glucose in DMR hydrolysate. Overall, slightly higher HMF yields were obtained with dehydration of glucose in DMR hydrolysate when compared to the HMF yields obtained with pure glucose. As shown in FIG. 9A, maximum xylose conversion approaching 93% was obtained which was lower than the maximum xylose conversion of 98% achieved with pure xylose. Moreover, a slightly lower furfural yields were obtained with dehydration of xylose in DMR hydrolysate when compared to the furfural yields obtained with dehydration of pure xylose though the highest furfural yield of 98% was still being achieved with the zeolites, i.e., H-ZSMS and H-BEA (FIG. 9B). Further optimization of reaction conditions for dehydration of biomass hydrolysate resulted in further increase in furfurals yield, i.e., 96% furfural and 74% HMF yields resulting in a combined furfurals yield of 80% at 197° C. for 5 min. Overall, among all the catalysts tested, amorphous silica-alumina (ASA) catalysts showed superior selectivity and productivity of furfurals compared to other solid catalysts evaluated such as zeolites and metal oxides.

Scheme 2 depicts a reaction scheme for dehydration of glucose or fructose to HMF catalyzed by Lewis and Brønsted acid catalysts and NaCl.

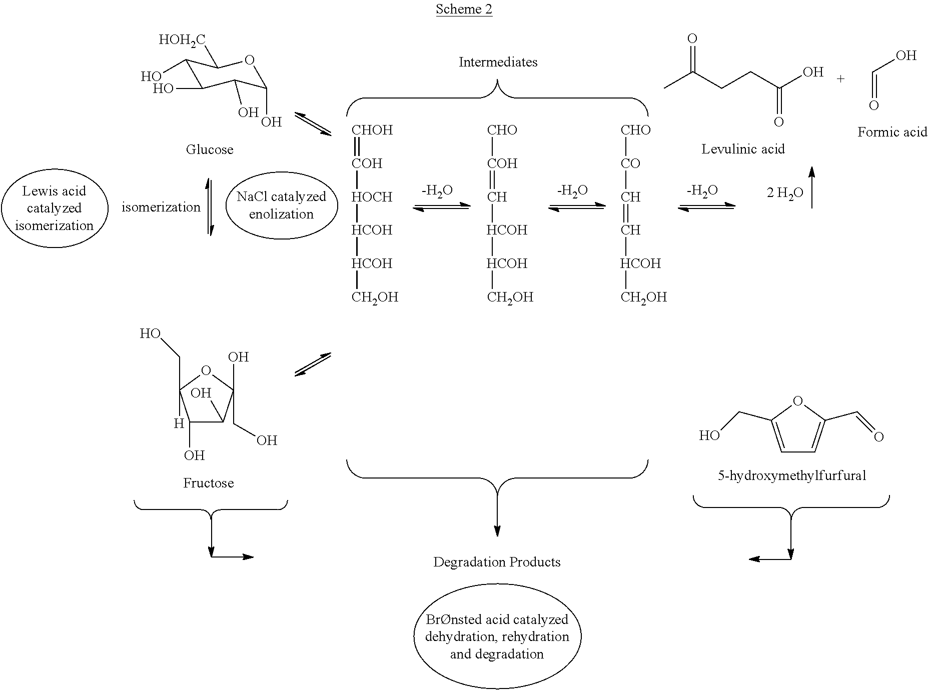

Insights into the roles of Lewis and Brønsted acid catalysts and NaCl in dehydration of glucose to HMF.

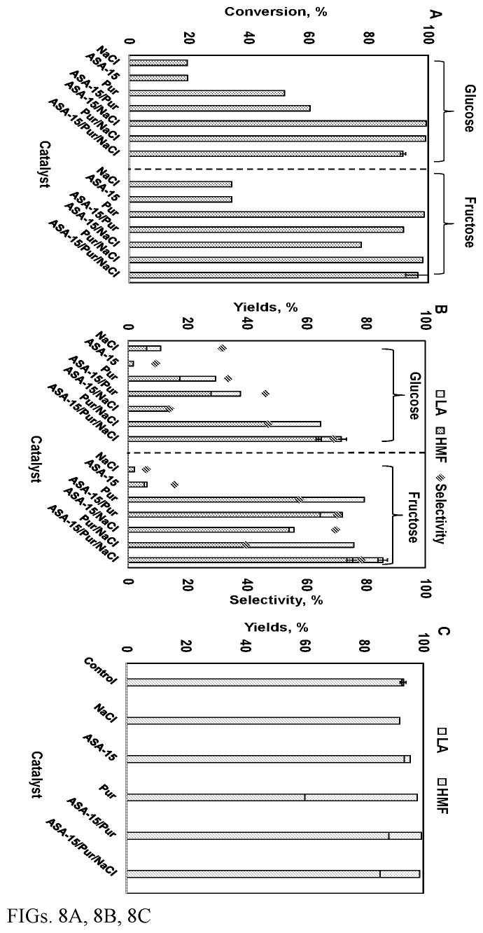

To gain insights into the roles of addition of Lewis and Brønsted acid catalysts and NaCl in dehydration of glucose to HMF, dehydration experiments were conducted with 8 wt % glucose, 8 wt % fructose and 4 wt % HMF at 195° C. and 5 min using dioxane: water— 2:1 (v/v) with or without the addition of 33 mM NaCl to ASA-15 and Pur-275 added either individually or together. A reaction scheme for dehydration of glucose or fructose to HMF catalyzed by Lewis and Brønsted acid catalysts and NaCl is shown according to the mechanisms proposed previously (Scheme 2). The conversion of glucose and fructose, HMF and LA yields from dehydration of glucose and fructose, and HMF yields obtained after reacting HMF under the same reaction conditions are shown in FIG. 8 and HMF yields are summarized in Table 2. From FIGS. 8A and 8B and Table 2, it can be noted that NaCl or the Lewis acid catalyst (ASA-15) alone cannot efficiently catalyze the dehydration of glucose or fructose to HMF. However, when both are added together, a remarkable 8- and 10-fold increments in HMF yields to 14 and 54%, for dehydration of glucose and fructose, respectively were obtained.

On the addition of the Brønsted acid catalyst (Pur-275) alone, a more than two-fold and three-fold increases in glucose (52%) and fructose (98%) conversions resulting in the HMF yields of 18% and 57%, respectively were obtained. The vastly different HMF yields obtained from dehydration of fructose and glucose with a Brønsted acid catalyst in this work corroborates the previously reported observation where high HMF yield from dehydration of the ketose form is achieved possibly due to its much faster enolization when compared to the aldose form. The addition of 33 mM NaCl to the Brønsted acid catalyst resulted in about 3-fold increase in HMF yield (47%) from the dehydration of aldose sugar showing that NaCl enhances the enolization of the aldose sugar aiding in its efficient dehydration to HMF (Scheme 2). Surprisingly, the HMF yield obtained with fructose decreased to 39% and LA yield increased from 22 to 37% due to increased rehydration reaction of HMF. The increase in rehydration of HMF observed in fructose dehydration here may possibly occur due to an increase in the concentrations of reactive intermediates leading to their side reactions under the more severe reaction conditions implemented here (195° C.) than generally needed for fructose dehydration (Scheme 2).

For the dehydration experiments conducted by adding catalysts with both Lewis and Brønsted acidity (ASA-15 and Pur-275) together, a slight increase in both conversion and HMF yield were obtained for dehydration of both glucose and fructose when compared to their conversions and HMF yields obtained with the addition of individual catalysts. Interestingly, on the addition of 33 mM NaCl to both the catalysts added together, both HMF yield and selectivity increased for dehydration of both glucose and fructose with the effect of NaCl addition being more noticeable for the dehydration of aldose form resulting in increase in HMF yields from 28 to 64% and 65 to 76% for dehydration of glucose and fructose, respectively. The significant increase in HMF yields from the dehydration of aldose form clearly suggests that NaCl promotes the rate of enolization of aldose form. The increase in HMF yields from the dehydration of ketose form suggests that NaCl possibly by promoting the rate of enolization of ketose as well as by stabilizing the reactive intermediates formed (Scheme 2).

Finally, to evaluate the effect of reaction severity and catalysts addition on HMF yield, experiments were conducted with 4 wt % HMF (FIG. 8C). The reaction of 4 wt % HMF at 195C/5 min with no catalysts added resulted in 93% recovery of HMF with a 7% loss to undetected compounds. Upon the addition of 33 mM NaCl, the recovery of HMF still remained at 92% showing that NaCl did not result in any additional degradation of HMF. Interestingly, no influence of the addition of ASA-15 was observed on HMF recovery, however, the addition of Pur-275 resulted in HMF recovery of only 60% with a significant increase in LA yield to 38% clearly showing that Brønsted acid catalyst catalyzes the rehydration reaction of HMF to LA and FA under the reaction conditions evaluated here. Surprisingly, with the addition of both Lewis and Brønsted acid catalysts together, HMF recovery increased to 88% with LA yield being reduced to 11%. Notably, the solution pH measured post reaction increased to 2.8 from below 2 with the addition of the Lewis acid catalyst suggesting that the presence of Lewis catalyst alters the solution pH and stabilizes or protects the HMF from its further degradation. On the addition of 33 mM NaCl to both Lewis and Brønsted acid catalysts, similar HMF recovery and LA yield were obtained again showing no detrimental effect of NaCl on HMF yield.

Overall, from the above experiments one can surmise that under the reaction conditions evaluated in this work, the addition of NaCl always resulted in increased HMF yield in dehydration of glucose resulting in maximum HMF yield when added to combined Lewis and Brønsted acid catalysts. However, in dehydration of fructose the effect of NaCl addition was more pronounced with Lewis acid catalysts with the maximum HMF yield still obtained when added to the combined Lewis and Brønsted acid catalysts. These results clearly show the promotional effect of NaCl by either enhancing the roles of each catalyst or stabilizing the reaction intermediates formed in dehydration of both aldose and ketose sugars to HMF.

Table 2 shows HMF yields obtained in dehydration of glucose, fructose, and HMF at 195° C./5 min with and without the addition of 33 mM NaCl to ASA-15 and Pur-275 added individually or together.

TABLE 2

| | Glucose (8 wt %) | | | Fructose (8 wt %) | | | HMF (4 wt %) | |
|---|---|---|---|---|---|---|---|---|
| | HMF yields, % | | Fold- | HMF yields, % | | Fold- | HMF yields, % | |
| Substrate | No NaCl | NaCl | change | No NaCl | NaCl | change | No NaCl | NaCl |
| ASA-15 | 1.8 | 13.9 | 7.7 | 5.4 | 54.2 | 10.0 | 93.5 | — |
| Pur-275 | 17.5 | 46.7 | 2.7 | 56.8 | 38.9 | 0.7 | 60.1 | — |
| ASA-15/Pur-275 | 27.9 | 64.1 | 2.3 | 64.6 | 75.6 | 1.2 | 88.4 | 85.4 |

In this work, various solid acid catalysts such as acidic ion-exchange resins, metal oxides, zeolites and amorphous silica-alumina catalysts were evaluated for simultaneous dehydration of mixed sugars present in biomass (DMR) hydrolysate to furfurals in a microwave reactor. A noticeable improvement in HMF yield from dehydration of 8 wt % glucose was obtained by combining solid acid catalysts with Lewis and Brønsted acidity resulting in HMF yields of 27-33% which increased by more than two-fold to 66-70% with the addition of NaCl in catalytic amounts (33-100 mM). Under the same reaction conditions, the dehydration of 6 wt % xylose resulted in furfural yields approaching 95%. Finally, simultaneous dehydration of 4 wt % xylose and 9 wt % glucose present in a process-relevant corn stover hydrolysate resulted in high yields of furfurals, i.e., 96% furfural and 74% HMF yields with a combined furfurals yield of 80% using an amorphous silica-alumina catalyst (ASA-15) and an acidic ion-exchange resin (Pur-275) in the presence of a catalytic amount of NaCl (33 mM) in a homogeneous solvent system of aqueous dioxane (dioxane: water— 2:1 v/v) at 197° C. for 5 min. For the various solid acid catalysts tested, the amorphous silica-alumina (ASA) catalysts showed superior productivity and selectivity of furfurals compared to other solid catalysts evaluated such as zeolites and metal oxides.

The approach developed in this work not only eliminated the need for a separate chemical or bio-catalytic isomerization of the aldoses (glucose and xylose) into their respective ketoses (fructose and xylulose) but also allowed to achieve their efficient dehydration to HMF and furfural simultaneously and presents a potential scenario of significant cost savings in the production of these important fuel precursors directly from biomass hydrolysates using heterogenous solid acid catalysts.

Methods

Raw Material

Glucose, fructose, xylose, furfural, HMF, AlCl₃.6H₂O, HCl, and Dioxane were purchased from Sigma-Aldrich. Pur-275 and Naf-50 were purchased from. TiO₂, ZrO₂, WO₃ and Nb₂O₅ were purchased from Sigma-Aldrich. (Davicat 3115 (ASA-15) and Davicat 3125 (ASA-25) were purchased from W. R. Grace & Co. TiO₂-SiO2, H-ZSMS and H-BEA were prepared according to the method reported previously (Ref —Dan). A process-relevant mixed sugar hydrolysate used in this study was obtained from corn stover using NREL-developed deacetylation and mechanical refining (DMR) process. The compositional analyses of biorefinery hydrolysates were conducted according to a standard Laboratory Analytical Procedure (LAP). Accordingly, the concentrations of monomeric and oligomeric sugars present in the hydrolysate were determined via 4% sulfuric acid hydrolysis treatment of the hydrolysate at 121° C. for 1 h. The glucose and pentose concentrations were determined to be 9 wt % and 4 wt %, respectively with about 7% of the sugars in the hydrolysate present as oligosaccharides.

Catalyst Preparation and Characterization

Total acid site density was determined using ammonia temperature-programmed desorption (NH3-TPD) with an Altamira AMI-390 Instrument with gas flow rates of 25 sccm. Catalyst samples (ca. 200 mg) were dehydrated in flowing Ar at 5° C./min to 450° C. for 4 h. The samples were cooled to 120° C. in flowing He, and then saturated with flowing 10% NH₃/He for 30 min. Excess and/or physisorbed NH₃ was removed with flowing He at 120° C. for 1 h. TPD of NH₃ was performed by heating the sample from 120 to 450° C. at 30° C./min and holding at 450° C. for 30 min. Desorbed NH₃ was measured with a TCD, and calibration was performed after each experiment by introducing 10 pulses of 10% NH₃/He through a 5 mL sample loop into a stream of flowing He.

The ratio of Brønsted to Lewis (B:L) acid sites was determined by diffuse reflectance infrared fourier transform spectroscopy of adsorbed pyridine (Py-DRIFTS) using a Thermo Nicolet iS50 FT-IR spectrometer operating at 4 cm⁻¹ resolution with a Harrick praying mantis attachment and CaF₂ windows operated at ambient pressure. The sample was dehydrated in flowing N2 at 300° C. for 4 h (100 sccm, 10° C./min ramp) and then cooled to 150° C. The sample was held at 150° C., and saturated pyridine vapor was introduced with 100 sccm of N2 for 10 min. Metal oxide samples were heated to 200° C. for 1 h and zeolite samples were heated to 300° C. for 1 h under flowing N2 to remove excess and/or physisorbed pyridine, cooled to 150° C. and spectra were collected. The absorption peaks near 1545 cm⁻¹ (Brønsted) and 1445 cm⁻¹ (Lewis) and their relative absorption coefficients (εB/εL=0.76) were used to determine the relative B:L acid site ratios.

Sugar Dehydration Reaction Experiments

Dehydration experiments were performed according to the method reported earlier.15 Briefly, reactions were conducted in batch mode at temperatures ranging from 185 to 200° C. using a Discover S-Class microwave-heated reactor (CEM Explorer, Matthews, NC). 10 mL glass reactor tubes filled to a liquid volume of 2-3 mL were used to conduct the reactions. After adding Lewis and Brønsted solid acid catalysts to the reaction tube, sugar solution (8 wt % glucose, 6 wt % xylose, or DMR hydrolysate) with or without the addition of NaCl at predetermined concentrations was added in the reactor tube followed by the addition of the organic solvent. The reproducibility of experiments was determined by conducting experiments in triplicates at various reaction times within each series of experiments.

Analyses of sugars (glucose, xylose and arabinose), FA and LA, and furfural and HMF were performed with a High-Performance Liquid Chromatography (HPLC) system (Agilent 1100, Agilent Technologies, Palo Alto, Calif.) according to the method reported elsewhere.15 Briefly, unreacted sugars and degradation products, i.e., FA and LA, were analyzed by HPLC using an Aminex HPX-87H Ion Exclusion column with a RID detector. Furfurals were analyzed using Reversed Phase HPLC analysis (RP-HPLC) using a Waters C18 reversed phase column. Furfural and HMF peaks were identified and quantified using a photodiode array detector (DAD).

Thus, in an embodiment, disclosed herein are methods for simultaneous dehydration of xylose and glucose present in biomass (DMR) hydrolysate to furfurals using solid acid catalysts in unprecedented high yields, i.e., 96% furfural and 74% HMF resulting in a combined yield of 80% furfurals, was achieved under mild reaction conditions (197° C., 5 minutes) in a microwave reactor. This is the highest reported combined furfurals yield from mixed sugars present in a biomass hydrolysate using solid acid catalysts (an amorphous silica-alumina catalyst (Davicat 3115, W. R. Grace & Co.) and an acidic ion-exchange resin (Purolite CT-275) in the presence of a catalytic amount of NaCl (33 mM).

The foregoing disclosure has been set forth merely to illustrate the invention and is not intended to be limiting.

We claim:

1. A method for the simultaneous dehydration of xylose and glucose in biomass hydrolysate to furfurals comprising the step of contacting the biomass hydrolysate with solid acid catalysts; and further comprising the step of contacting the biomass hydrolysate with a silica-alumina catalyst in the presence of a catalytic amount of NaCl; and further comprising contacting the biomass hydrolysate with an acidic ion-exchange resin in a homogeneous solvent system of aqueous dioxane.

2. The method of claim 1 wherein the solid acid catalyst is selected from the group consisting of acidic ion-exchange resins, metal oxides, zeolites and amorphous silica-alumina catalysts catalyst.

3. The method of claim 2 wherein the silica-alumina catalyst is Davicat 3115.

4. The method of claim 1 wherein the yield of furfurals from the biomass hydrolysate is about 96% or greater.

5. The method of claim 1 wherein the yield of furfural is 5-hydroxymethylfurfural and is greater than about 74%.

6. The method of claim 1 wherein the yield of combined furfurals is greater than about 80%.

7. The method of claim 1 wherein the catalytic amount of NaCl is about 33 mM.

8. The method of claim 1 wherein the acidic ion-exchange resin is Purolite 275-CT.

9. The method of claim 1 wherein the aqueous dioxane solvent system consists of a mixture of 2 parts dioxane to 1 part water (vol/vol).

10. The method of claim 1 wherein the dehydration reaction is at about 197 degrees Celsius.

11. The method of claim 10 wherein the dehydration reaction is for about 5 minutes.

12. The method of claim 10 wherein the dehydration reaction is in a microwave reactor.

13. A method for simultaneous dehydration of glucose and xylose present in a biomass hydrolysate to furfural and 5-hydroxymethylfurfural using a heterogenous solid acid catalyst in a microwave reactor.

14. The method of claim 13 wherein the solid acid catalyst is selected from the group consisting of acidic ion-exchange resins, metal oxides, zeolites and amorphous silica-alumina catalysts catalyst.

15. The method of claim 13 wherein the dehydration is at about 197 degrees Celsius.

16. The method of claim 13 wherein the dehydration reaction is for about 5 minutes.

17. The method of claim 13 wherein the biomass hydrolysate is corn stover hydrolysate.

18. The method of claim 17 wherein the yield of furfural is 96%.

19. The method of claim 17 wherein the yield of furfural is 5-hydroxymethylfurfural and is greater than about 74%.

20. The method of claim 17 wherein the yield of furfurals is greater than about 80%.
